(19) United States
(12) Patent Application Publication
(10) Pub. No.: US 2011/0041852 A1
(43) Pub. Date: Feb. 24, 2011

(54) AMBU-BAG AUTOMATION SYSTEM AND METHOD

(76) Inventor: Robert T. Bergman, Columbus, IN (US)

Correspondence Address: Woodard, Emhardt, Moriarty, McNett & Henry LLP, 111 Monument Circle, Suite 3700, Indianapolis, IN, 46204-5137, US

(21) Appl. No.: 12/545,467

(22) Filed: Aug. 21, 2009

(51) Int. Cl. A61M 16/08

(52) U.S. Cl. 128/205.13

(57) ABSTRACT

A device for automatically squeezing and releasing an AMBU-bag is disclosed. A device has a housing, and a mechanical compression squeezer in the housing. There are openings in the housing for inlet tubes and outlet tubes of AMBU-bag to pass in and out of the housing. A powered actuator powers the compression squeezer.

### FIELD OF INVENTION

The present invention relates to AMBU-bags, and more specifically, a system for automatically squeezing and/or releasing of an AMBU-bag.

### BACKGROUND

AMBU-bags are in wide spread use in medical and emergency treatment of patients. They are designed for manually squeezing, such as by a doctor, nurse, orderly, EMT or other medical service provider. Their usage includes, for example, respiration a patient (civilian or soldier) in the field and/or during transport to a hospital. Their usage also includes keeping a patient respirated during movement from one location to another. For example, an AMBU-bag may be used for a patient being transported on a gurney from their hospital room (where they are ordinarily hooked up to a respirator) to a surgical operating room, where they will be hooked up on a second respirator in the operating room. The AMBU-bag is typically manually operated during such movement of a patient. Otherwise, patients needing respiration are typically hooked up to a respirator. This device may be used to supplement limited inventories of respirators, such as in the case of an epidemic or other high demand.

### SUMMARY

The claims, and only the claims, define the invention. The present invention includes several, but not necessarily all, of a device for use with an AMBU-bag, the AMBU-bag having a flexible squeeze bag with in intake tube and first end outlet tube at an opposite end thereof. The device may have a housing for receiving the squeeze bag. The device may have an opening for the AMBU-bag narrow intake tube, and another one optionally for the AMBU-bag air and outlet tube.

A mechanical compression squeezer is in the housing for cyclically squeezing a squeeze bag from its outside and releasing the squeeze for expansion. The power actuator, controlled by an electronic timer, is for powering the mechanical compression squeezer for cyclical squeezing.

Other optional features that may be included, but are not required, are set forth in the various dependent and independent claims.

### BRIEF DESCRIPTION OF THE DRAWINGS

FIG. 1A is front view of the device according to one example of the present invention with an optional lid closed, and with no AMBU-bag in it.

FIG. 1B shows the device in FIG. 1A with the lid open.

FIG. 1C shows the device of FIG. 1B with an AMBU-bag in the housing.

FIG. 1D shows the device of FIG. 1C with the lid closed.

FIG. 2A is a side view of the device of FIG. 1A.

FIG. 2B is a side view of the device of FIG. 1B.

FIG. 2C is a side view, partially cut away, of the device of FIG. 1C.

FIG. 2D is a side view, partially cut away, of the device of FIG. 1D.

FIG. 3 is a side view opposite the side shown in FIG. 2A.

FIG. 4 is a bottom view of the device of FIG. 3.

FIG. 5 is the top view of the device of FIG. 3.

FIGS. 6A-6G illustrate various examples of mechanical compression squeezers.

FIG. 7A is a partial cut away view showing a recessed hook.

FIG. 7B shows the device of FIG. 7A with the hook out of the recess hooked and on a bedrail.

FIG. 8 is an alternative version of the hook shown in FIG. 7B.

FIG. 9 is one example of a schematic view (not to scale) of optional electronics.

### BRIEF DESCRIPTION OF THE PREFERRED EMBODIMENTS

For the purposes of promoting an understanding of the principles of the invention, reference will now be made to the examples, sometimes referred to as embodiments, illustrated and/or described herein. Those are mere examples. It will nevertheless be understood that no limitation of the scope of the invention is thereby intended. Such alterations and further modifications in the described processes, systems or devices, any further applications of the principles of the invention as described herein, are contemplated as would normally occur to one skilled in the art to which the invention relates, now and/or in the future in light of this document.

As used in the claims and the specification, the following terms have the following defined meanings:

The term “AMBU-bag” means an ambulatory or movable bag that is manually squeezable to provide or assist in respiration of a patient.

The term “blood pressure meter” means any meter, electronic, hydraulic, mercury based or otherwise that measures blood pressure of a patient.

The term “clean” means substantially free of germs and/or pathogens sufficient for medical and/or surgical exposure to a patient's lungs of gas passing through.

The term “CO₂ meter” means any meter that measures the amount of carbon dioxide exhaled by a patient.

The term “compression squeezer” means a mechanical, electromechanical, (and/or unless denoted “non-pneumatic/hydraulic”), pneumatic and/or hydraulic) component that provides force and/or pressure on an outside of an AMBU-bag to squeeze it to cause air/gas to flow through it.

The term “confine” means substantially restrict or hold in place.

The term “controlled” means controlled by an operator and/or computer processor to achieve a result or output.

The term “cycle frequency” means the frequency, typically measured in breaths per minute, of squeezing and releasing an AMBU-bag.

The term “cyclically” means a repetitious cycle, typically although not always of a consistent frequency.

The term “detachment of said intake tube” means operator separation of intake tube, directly or indirectly, from the remaining portion of the AMBU-bag.

The term “detachment of said outlet tube” means operator separation of outlet tube, directly or indirectly, from the remaining portion of the AMBU-bag.

The term “distal attachments” means various tubes, hoses, patient mouth pieces, masks, and/or patient tracheotomy attachments, attached from the outlet of an AMBU-bag.

The term “drop-in insertion” means the inserting of an AMBU-bag into place without requiring substantial mechanical disassembly and/or assembly.

The term “electronic blood-oxygen level sensor” means a sensor of a patient's blood oxygen level that takes an output and transforms in into an electronic signal which is then translated into a numeric and/or alpha numeric indicator of blood oxygen level. This can include, but is not limited to, sensors utilizing light or other transmissive frequency through a finger or other body part to provide input data to determine blood oxygen level.

The term “electronic timer” means a timer, typically in seconds, which is electrical rather than mechanical.

The term “end” means one or either ends of an elongated structure, such as for example an elongated AMBU-bag or an elongated housing, as opposed to its sides.

The term “expansion” means to increase volume.

The term “end member” means a structure at or near one end of the AMBU-bag. It may be rigid, flexible, or both.

The term “fixed portion” means substantially rigid or substantially immovable with respect to the housing when in use.

The term “flexible” means bendable or pliable to allow expansion and squeezing.

The term “flexible tension member” means one or more flexible belts, straps, cables, cords or the like.

The term “holding” means maintaining and/or capable of substantially maintaining something in position with respect to something else.

The term “hooks” means mechanical structure strong enough to hang the housing from.

The term “hoop stress” means circumferential loading around all, or more typically a portion, of the bag for causing squeezing of the bag.

The term “housing” means an outer case, shell, frame, grid, or structure. It may be partially or wholly solid material, mesh or cage structure, and/or both. It may be made from a variety of materials, although metal and/or rigid plastic are preferred. It may be opaque, transparent, translucent, and/or a combination of the above.

The term “intake tube” means a tube or conduit (regardless of cross section, round, square, rectangle, oblong or otherwise) which may be rigid, flexible, and/or both, in which is attached to or near the intake of the bag.

The term “magnitude of squeezing” means the amount of squeezing into the AMBU-bag, corresponding (directly and/or non-linearly) to the amount of air/gas flowing through the bag.

The term “mechanical” means other than by human muscle and/or bone and/or exhalation force.

The term “movable rigid member” means one or more rigid members that are designed to move, either by translation, pivoting or otherwise, with respect to the housing. The rigidity may be variable, but preferably substantially rigid.

The term “near” means close enough to functionally achieve inter-operability between two parts, directly, indirectly and/or with or without one or more intervening parts.

The term “open-close lid” means a lid which has at least two positions, one position being open and the other position being closed.

The term “opening” means one or more holes, slots, apertures, and/or slots in a member, wall, mesh, cage, or the like.

The term “operator adjustment” means capable of being adjusted or modified by the operator.

The term “opposite” means opposed to or generally across from.

The term “outlet tube” means a tube or conduit (regardless of cross section, round, square, rectangle, oblong or otherwise) which may be rigid, flexible, and/or both, in which is attached to the outlet of the bag.

The term “outside” means not from within. For example, the outside of an AMBU-bag means the outer portion which is squeezed, as opposed to the inner surface of a bag.

The term “portable” means sufficiently light and small that it can be carried by a single adult human.

The term “powered actuator” means a mechanism that provides movement other than by human power. This includes, but is not limited to electrical power, mechanical power, hydraulic power, pneumatic power, and/or a combination thereof. Frequently, but not necessarily a powered actuator includes one or more electrical motors.

The term “receiving” means taking or being capable of taking one thing within (partially or wholly) or in engagement or connection with another.

The term “recessable” means partially and/or wholly receiving one component within a recess. Preferably, one something that is recessable it is fully flushed or below fully flushed with respect to a reference surface, although optionally can include being partially recessed.

The term “releasing” means the opposite of squeezing.

The term “running” means from one location to another location.

The term “slot” means an opening that is general longer than it is wide. A slot may be linear, curved, serpentine or otherwise, and correspondingly the length of the slot would be linear, curved and/or serpentine.

The term “squeeze bag” means a portion of an AMBU-bag that may be squeezed and released to cause air/gas flow.

The term “volume controller” means a knob, slide, key pad, or other user input and associated electrical components to increase, decrease and/or maintain the amount air/gas squeezed through the squeeze bag.

The term “volume of air/gas per cycle” means the amount of air/gas, typically expressed in cubic inches per minute, cubic inches per second, cubic liters and/or centiliters per minute and/or per second for a given squeeze and release for a squeeze bag.

The term “yoke” means a mechanical structure, typically alone or in combination with other parts confining on three or four sides of another member or tube. Optionally, the yoke may be capped or uncapped, such that the fourth, optionally open side of the yoke may be closed or capped. The yoke may be rigid or flexible, but preferably it is rigidly or substantially rigid. The yoke may be lined or unlined with softer material, friction increasing material (such as rubber or otherwise). The yoke may be of variety of shapes, including rounded, rectilinear, or otherwise. A yoke may be defined, in whole or in part, by the edge of a slot or other opening.

Articles and phases such as, “the”, “a”, “an”, “at least one”, and “a first”, are not limited to mean only one, but rather are inclusive and open ended to also include, optionally, multiple such elements. Likewise, “comprising” is open ended and inclusive.

Referring to the drawing figures, these are only examples of the invention, and the invention is not limited to what is shown in the drawings.

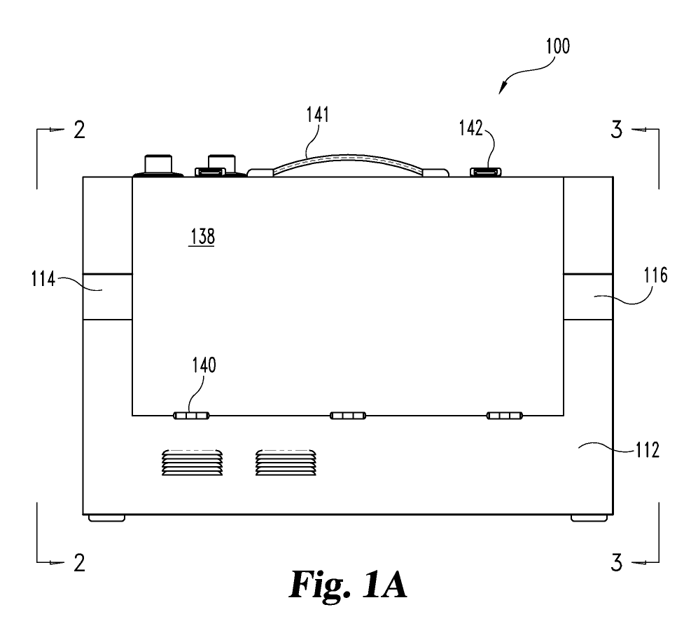

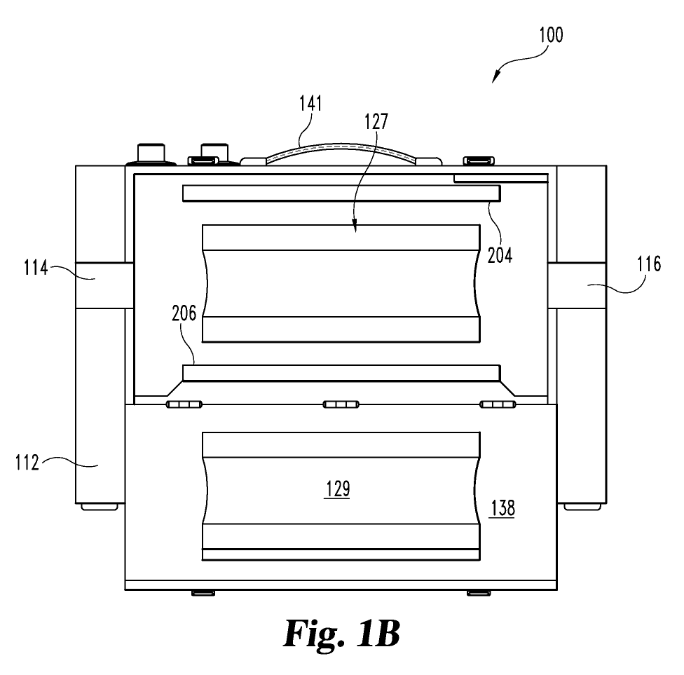

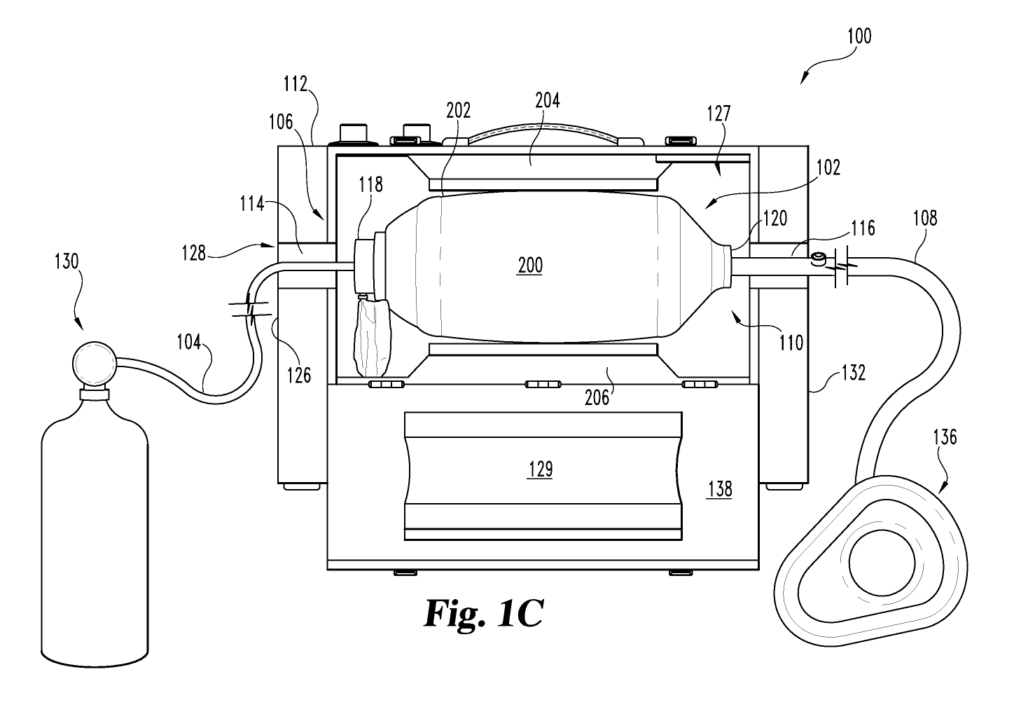

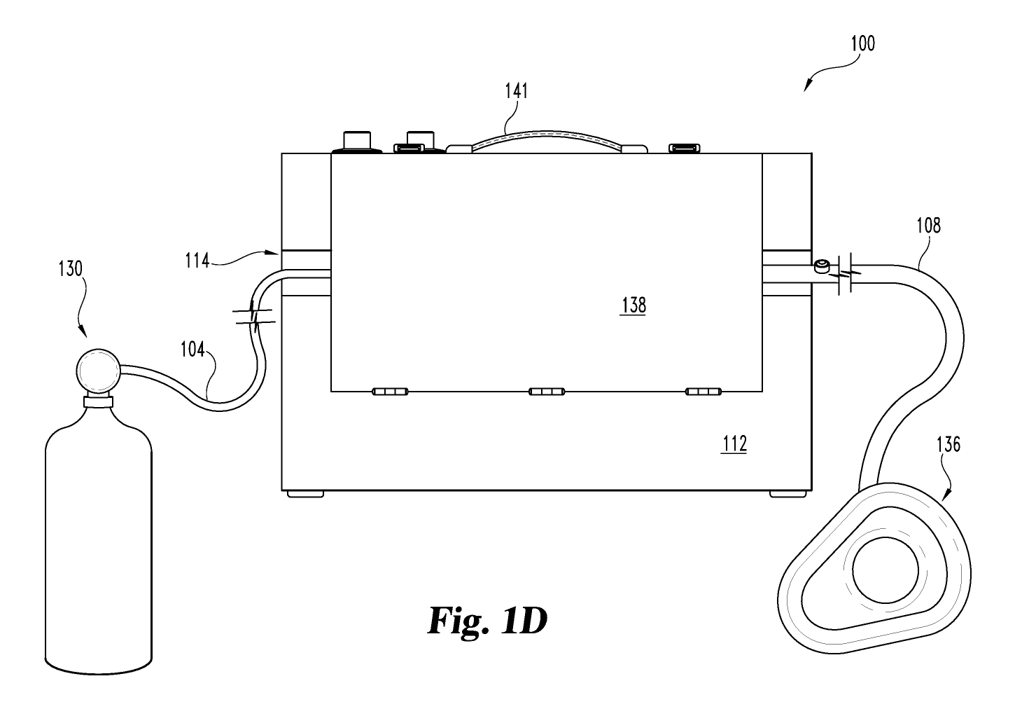

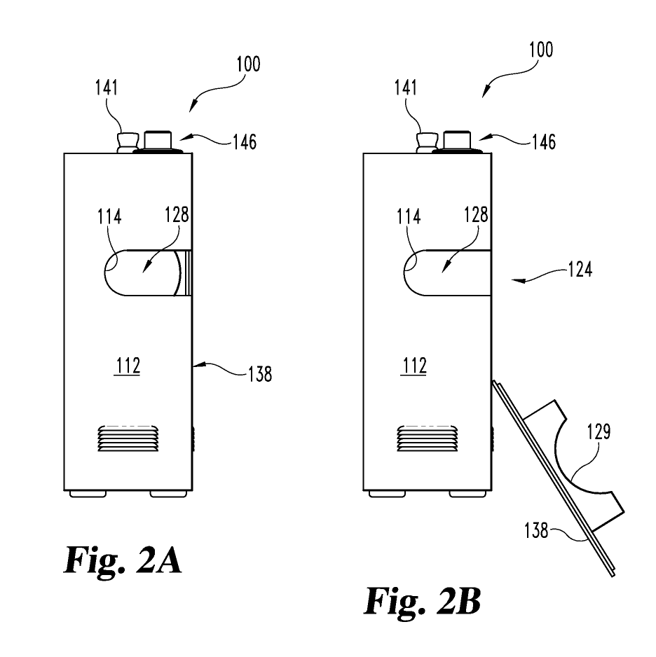

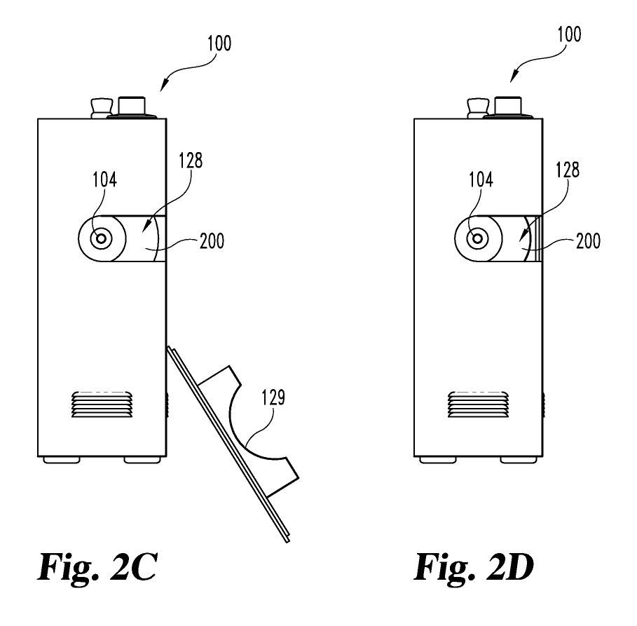

As but an example of the device, device 100 is for use with the AMBU-bag 102 having a flexible squeeze bag 200. Ordinarily, the AMBU-bag is initially clean, particularly on its inner surfaces that contact air/gas to the patient; and, it may optionally be a one-time use or disposable product. Preferably, the squeeze bag has an intake tube 104 (shown with broken lines) at a first end 106 of the AMBU-bag, and an outlet tube 108 (shown with broken lines) at an opposite end 110 of the AMBU-bag. Device 100 preferably comprises a housing 112 for receiving squeeze bag 200 (see FIG. 1C). In one example, the housing is generally rectangular, preferably elongated, although it may be any shape (square, cylindrical, or otherwise).

Optionally, the bottom of housing 102 may include several rubber feet, such as rubber feet 103 (see FIG. 4). Feet may be located elsewhere, such as opposite opening 124. Also, as illustrated in the various figures, optionally ventilation slots and/or louvers may be provided to facilitate cooling of the electronics, battery and/or actuators.

Optionally, but preferably, first yoke 114 may be on the housing for holding the AMBU-bag near intake tube 104. Also, optionally, a second yoke 116 may be provided on the housing for holding the AMBU-bag near outlet tube 108.

Note that as illustrated in FIG. 1C, this one particular example shows yoke 114 and 116 holding inlet tube 104, and outlet tube 108 respectively. However, optionally, one or more of these yokes may be positioned and/or shaped closer to the AMBU-bag such that the yoke partially or completely holds collar 118 and/or collar 120. Further optionally, such yoke may hold the AMBU-bag partially axially inward of such collars and/or a combination of holding the inlet and/or outlet tubes, collars, and locations inboard thereof, alone or in combination. Moreover, the yoke does not necessarily have to co-exist with an opening in the housing, but rather may be a separate structure. For example, the yoke may be a separate structure inside the housing, with an associated opening to allow ingress of tube 104 and egress of tube 108.

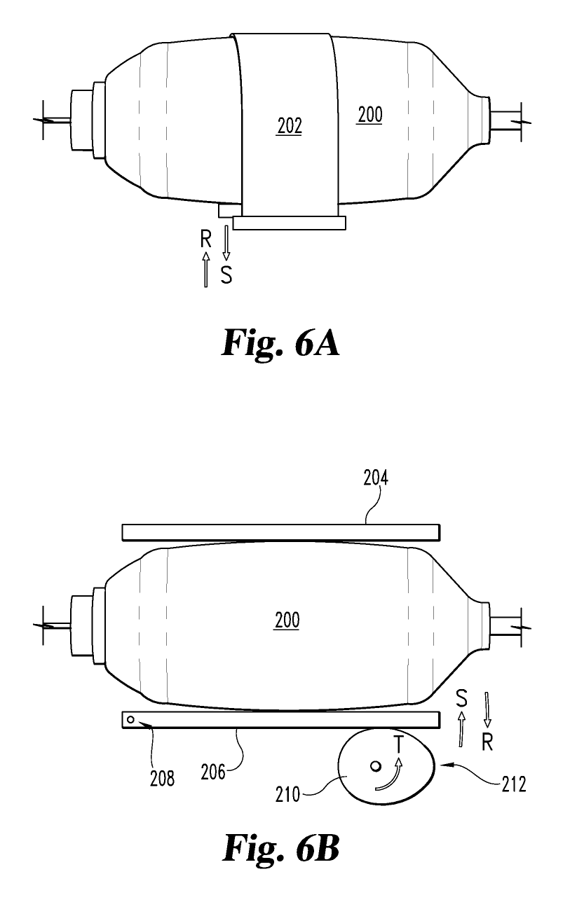

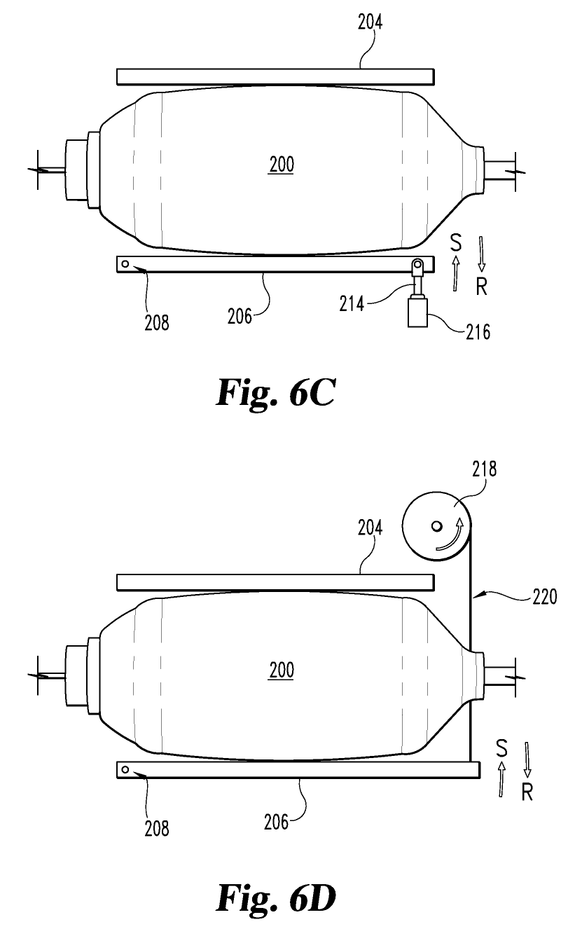

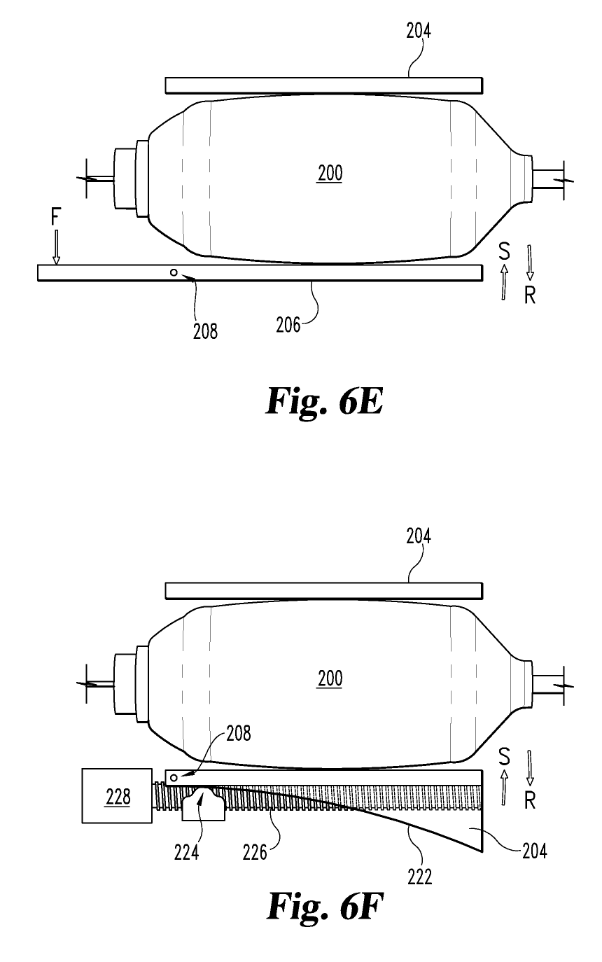

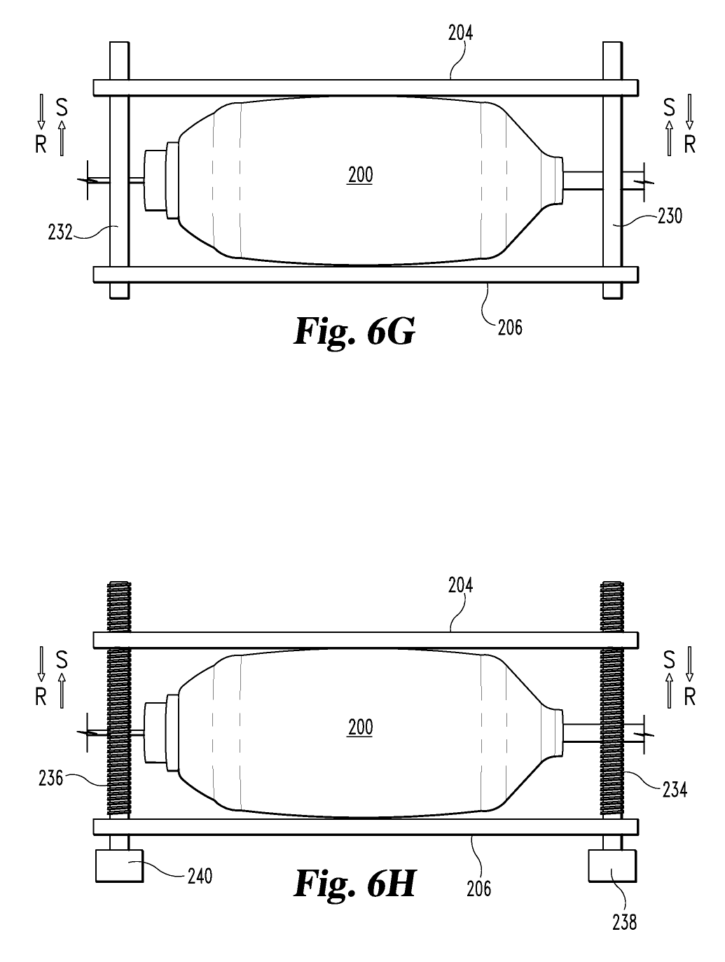

Preferably, one or more mechanical compression squeezers are part of device 100, preferably in housing 112. Mechanical compression squeezers, as defined herein, can be of a variety of configurations. As mere examples, various mechanical compression squeezers are illustrated in FIGS. 6A-6G, discussed further below. The mechanical compression squeezer (and/or squeezers) are for cyclically squeezing squeeze bag 200 from its outside 202 (see FIG. 1C) and releasing the squeeze bag for expansion. Note that FIG. 1C illustrates the squeeze bag in an expanded state.

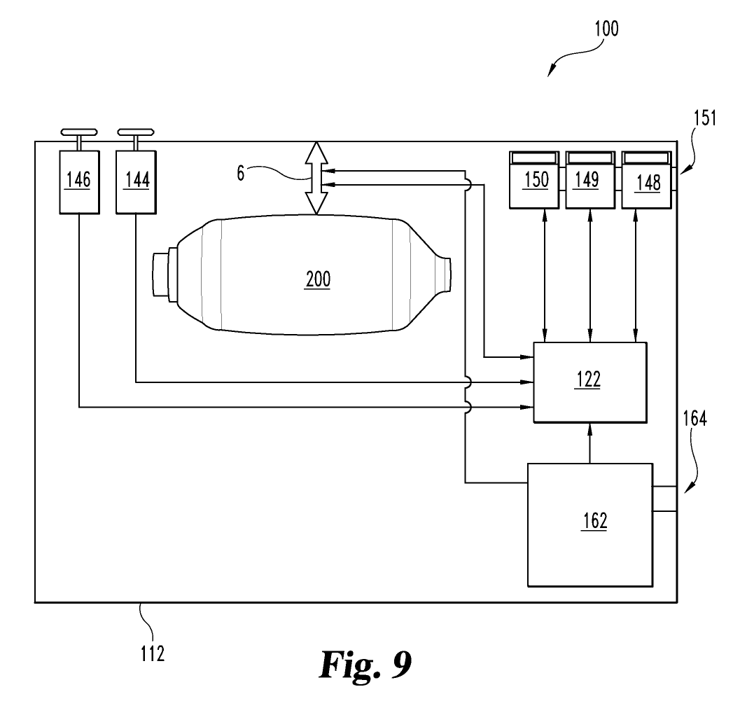

Typically, a powered actuator is controlled by an electronic controller and/or timer 122 (see for example FIG. 9) is for powering the mechanical compression squeezer for this cyclical squeezing.

Preferably, there is an opening 124 in the housing, wherein the opening allows drop in insertion of this squeeze bag within the housing. For example, opening 124 is illustrated in FIG. 1B prior to insertion of the squeeze bag, whereas FIG. 1C shows opening 124 after the squeeze bag 200 has been dropped within the housing.

Optionally, a first end member 126 has a first slot or other opening 128, preferably running from the opening 124 in the housing to allow drop in insertion of the AMBU-bag without requiring detachment of the intake tube 104 from distal attachments 130 to the intake tube 104. Optionally, but preferably, such drop in insertion in slot 128 likewise allows drop in insertion of the AMBU-bag in the housing without requiring detachment of the intake tube from the AMBU-bag.

Optionally, a second end member 132 has a second slot or other opening 134 running from the opening 124 in the housing to allow drop in insertion of the outlet tube 108 without requiring detachment of the outlet tube from distal attachments 136. Again, optionally, slot 134 may allow drop in insertion of the outlet tube 108 without requiring detachment of the outlet tube from AMBU-bag 102.

Optionally, an open-closed lid may be provided at opening 124 wherein the lid is openable to allow drop in insertion of the squeezed bag within the housing and is closeable to confine the squeeze bag within the housing 102. When an open-closed lid is used, it may be a solid material, mesh, bars, opaque, transparent, or otherwise. In the one example illustrated, it may include hinges such as hinge 140 and/or latches such as latch 142, although these are optional. Furthermore, while opening 124 and/or lid 138 are, in this one illustrated example, shown on the front of the housing, they may be located elsewhere. For example, they may be located on top, bottom, back or side of the housing as well as a combination thereof. For example, the opening may effectively wrap around two or more sides of the housing. Likewise, optionally the lid may wrap around such an opening.

Optionally, one or more confinement members, such as confinement members 127 and/or 129 may be included to help confine bag 200. In the examples shown, member 129 is on the lid 138 and when closed may confine a bag on member 127. They optionally may have partially curved surfaces, such as portions of a cylinder shaped surface as illustrated, but also if used may have other shapes as well.

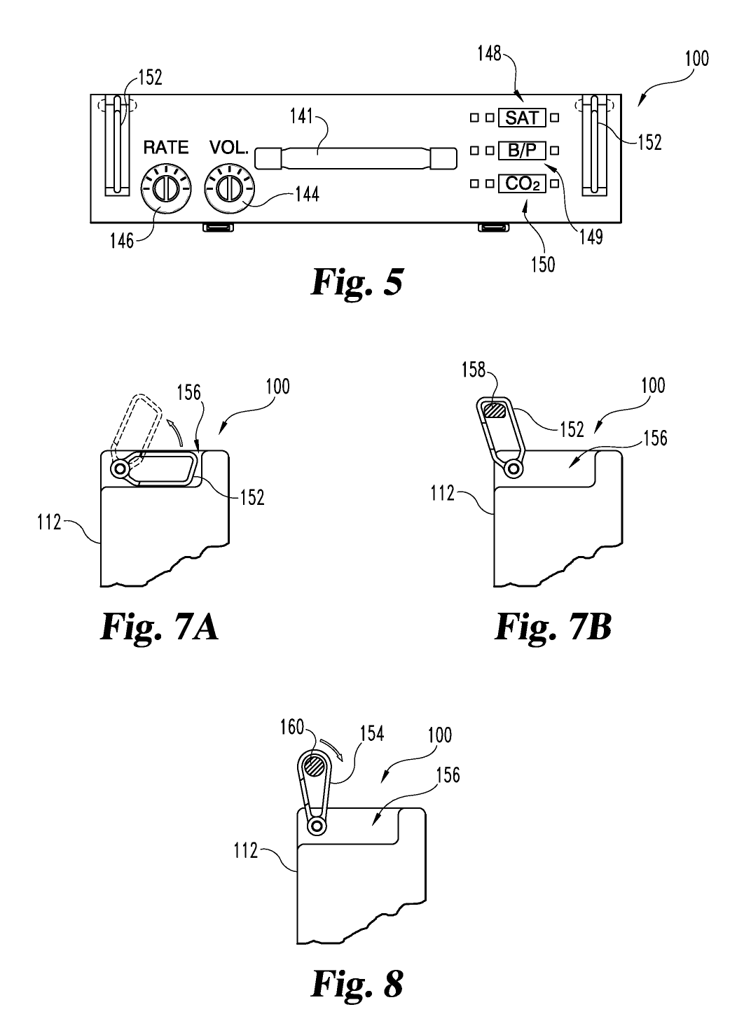

With reference to FIG. 5, an optional adjustable volume controller 144 may be included providing operator adjustment of the magnitude of the squeezing of the mechanical compression squeezer to adjust the volume of air/gas per cycle through AMBU-bag 102. Such controller is also illustrated schematically in FIG. 9.

Furthermore, the optional feature of an adjustable cycle controller 146 may be included, providing operator adjustment of cycle frequency of the mechanical compression squeezer (see FIGS. 5, 9).

Also referring to FIGS. 5 and 9, one or more of optional built in electronic blood-oxygen level sensor 148, CO₂ meter 150, and/or blood pressure meter 149, including read out(s) be provided as well.

Another optional feature is the use of mounting structures, including without limitation hooks. For example, preferably these may include recessed hooks adapted to hang the housing 102 off a hospital bedrail. For example, with reference to FIGS. 7A, 7B and 8, recessable hooks 152 and 154 are illustrated. These may be recessed (See FIG. 7A) in a recess 156. These may be adapted to hang off of a hospital bedrail 158 or 160. As such, such hooks may be optionally comprise open hooks, or as illustrated spring loaded carabineer type hooks. Optionally, they may pivot on an axis to swing up for hooking on the bedrail or other structure (such as a hanging structure in a helicopter or ambulance, or otherwise), or alternatively be pivoted down into the recessed mode.

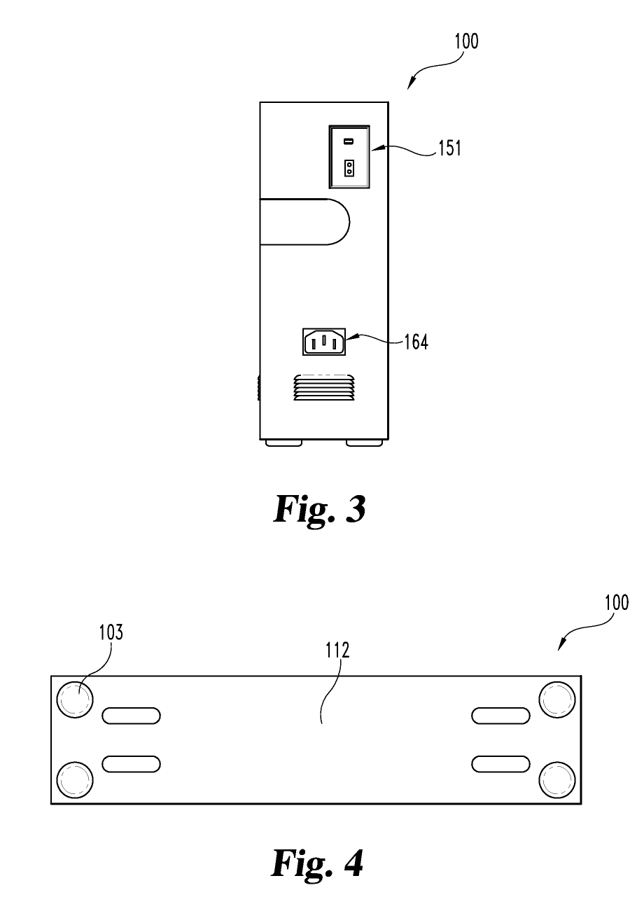

Preferably, the device 100 in the housing 102 is portable such that it will fit in a vehicle, such as a helicopter or ambulance, and optionally, but preferably, includes a battery 162 (see FIG. 9) for providing power to one or more power actuators. Note further that the battery, or other power source (such as AC and/or DC power) may provide power to controller/microprocessor 120 and other electrical components in the device. Note further that preferably, the battery is replaceable, preferably easily and quickly replaceable for field operations. Also, the battery may include a recharger including a recharger fitting 164 (see FIG. 3) in the housing.

FIG. 9 illustrates one example of a schematic layout of a portion of the illustrated device. Housing 102 may include squeeze bag 200. The one or more mechanical compression squeezers are diagrammatically illustrated as 6. Controllers 144 and 146 as previously described provide input, as indicated by the input arrows, to control controller/microprocessor 122.

Controller 122 may receive input and/or provide controlling output to squeezer 6, both in terms of cyclical frequency as well as squeezing volume, or both, or neither, as previously described. Optionally, one or more sensors may be placed on the compression squeezers and/or the outside service of the AMBU-bag. For example, such sensors may detect pressure and/or displacement. A pressure sensor may be used to correlate to the amount of back pressure in the patient's lungs. Similarly, displacement sensor may correlate to the volume of air pushed or forced into the patient's lungs. By providing such optional sensors and feedback (not illustrated in the drawings), the controller/microprocessor 122 may receive data input therefrom. Optionally, such data input may be used to fully or partially automate and/or self-adjust the amount of squeezing pressure and/or volume, such as to accommodate the various physiologies of various patients. Controller/microprocessor 122 may also provide input and/or output to blood oxygen, CO₂ meter and/or blood pressure meters 148, 149 and/or 150 as well. Optionally, feedback from the meters to the microprocessor/controller 122 may be used alone or in connection with pressure and/or volume feedback sensors and/or pressure and/or volume controllers, discussed above. For example, if the blood oxygen and/or CO₂ level for a patient, as detected, falls below the predetermined level, the controller/microprocessor 122 may be programmed to self-adjust to increase the cyclical frequency, thereby passing more air/gas through the patient's lungs, hopefully increasing the blood oxygen level to the desired threshold level.

Note also that the meters 148, 149 and/or 150 preferably receive their input via plug in fitting 151, whereby blood pressure detectors attach to the patient and/or blood oxygen level attach to the patient are plugged into device 100. Typically, the CO₂ measurement is take from or in tube 108 and/or attachment 136.

As mentioned, FIG. 6A-6G are merely examples of types of compression squeezers that may be utilized, as illustrated or as modified, that are combined with each other.

For example, FIG. 6A illustrates the use of one or more strap, such strap 202 wrapped wholly or partially around squeeze bag 200. Cyclical tension and/or pulling on strap 200 causes squeezing S, whereas converse releasing of such pulling/tension allows release R of the squeeze bag. Such pulling may be effectuated by any type of mechanical, pneumatic, hydraulic or other action, including motorized winders, reciprocated solenoids or other plungers, gears or the like.

Note that with respect to strap 202 in FIG. 6A, this may pull a hoop stress on the outside of the squeeze bag. Another alternative arrangement would be that member 202 could itself be an inflatable bladder, filled with compressed gas and/or liquid, thereby effectuating hoop stress and/or other squeezing on the squeeze bag. Note also that the member 202 may be preferably conveniently coupled and uncoupled to allow convenient drop in insertion of the squeeze bag within the housing, with subsequent confinement of the squeeze bag by wrapping the member 202 around it. Optionally, one or more handles, such as handle 141, may be provided on case 112, such as to make it more conveniently portable.

FIG. 6B illustrates an alternative embodiment in which squeeze bag 200 may be positioned between one or more members, optionally but preferably rigid. Note that one or more of such members may be stationery or moveable, although preferably at least one, if not both are movable. For example, as diagrammed in 6B, member 206 is moveable but pivoting around pivot 208. A rotating cam 210 is provided, which may rotate or turn in direction indicated. Cam 210 can include one or more lobe 212 which, when turned in engagement with member 206 causes squeezing S, whereas disengaging of lobe may allow releasing R. Note that optionally, but not required, member 206 may be biased, with springs, counter-rotation cams, or otherwise, in the released position. The same may be true of other examples described herein. Note although members 204 and/or 206, in this particular side of examples illustrated as shown separate from the housing, they may be wholly or partially part of the housing. For example, optionally member 204 may simply be a wall or portion of the housing itself. Note that although as illustrated, the axis of pivot 208 is transverse and skew to central longitudinal axis of AMBU-bag 102, it may be any orientation including parallel.

FIG. 6C illustrates member 206 moveable by a reciprocator, such as for example, having an extendable arm 214 movable (telescopically or otherwise) with respect to element 216. In such case, extension upward causes squeezing S as illustrated, whereas movement in the opposite direction causes pivoting of member 206 around pivot 208 back to the release R direction/position. Such reciprocate actuator can be electrical, solenoid, servo, warm gear, rack gear or other such operation, and preferably as before powered with a powered actuator such as a motor, hydraulics, pneumatics or otherwise.

FIG. 6D illustrates a version using a winder to a turn such as indicated by the arrow, winding, and thereby exerting tension on tension member, such as cable 220 or cord or otherwise. When it is wound in tension in the arrangement illustrated, member 204 pivots around 208 and squeezes in the direction S. When winder 218 releases, the bag may be released in direction R. Again, spring or biasing may be used, or alternatively in opposite directed counterwinding mechanism or cam or otherwise may be used to effectuate releasing R.

FIG. 6E illustrates the use of force F on an opposite side of pivot 208, to create a fulcrum action for squeezing S. As such, any of the previously or subsequently mentioned actuators, cams, tension members, and otherwise may be directed on the opposite side of pivot 208, and those situations when a pivot is used, to effectuate squeezing. Conversely, a direction opposite to force F may be used at the location indicated at force F to effectuate releasing R. Note further that all of the actuator mechanisms described in this document may be concurrently imparted on more than one member, such as being imparted on both member 204 and member 206 simultaneously.

FIG. 6F indicates an arrangement with a cam surface 222 and a cam follower 224. Linear movement of the cam follower (for example, left to right) exert force on cam surface 222, thereby causing member 204 to pivot around pivot 208, thereby effectuating the squeezing S. As one example, cam follower 224 may be moved longitudinally by having threaded engagement with the gear, such as warm gear 226 rotated by gear/motor driver 228. Other arrangements may be included, such as using a gear rack rather than a worm gear and/or gear driving and/or motorizing the follower 224.

While the foregoing examples include pivoting motion, such as pivoting 208, such pivoting is not required. For example, in FIG. 6G, squeezing and/or releasing may be effected by moving one or more of member 204 and/or 206 longitudinally, such as long guides 230 and 232. Squeezing and/or releasing force may be, for example, of the type previously described, and/or pneumatic and/or hydraulic inflation bags or the like.

Similarly, instead of sliding guides or rails, such as previously mentioned with respect to FIG. 6G, FIG. 6H describes an alternative arrangement in which gear drives, such as one or more of worm gears like worm gear 234 and/or 236 may be rotated, such as by rotating driver 238 and/or 240, thereby causing squeezing S and/or releasing R of bag 200.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. It is also contemplated that structures and features embodied in the present examples can be altered, rearranged, substituted, deleted, duplicated, combined, or added to each other.

What is claimed is:

1. A device for use with an AMBU-bag having a flexible squeeze bag with an intake tube and an outlet tube, comprising:
a housing for receiving said squeeze bag;
a first opening in said housing adapted for an intake tube of said AMBU-bag to connect with said squeeze bag while it is in said housing;
a second opening in said housing adapted for an outlet tube of said AMBU-bag to connect with said squeeze bag while it is in said housing;
a mechanical compression squeezer in said housing for cyclically squeezing said squeeze bag from its outside and releasing said squeeze bag for expansion; and, a powered actuator for powering said mechanical compression squeezer for said cyclical squeezing.

2. The device of claim 1, and further comprising:
a third opening in said housing, wherein said third opening allows drop-in insertion of said squeeze bag in said housing;
a first end member of said housing, having a first slot running from said third opening in said housing to allow drop-in insertion of said AMBU-bag without requiring detachment of said intake tube from distal attachments; and
a second end member of said housing, opposite said first end member, and having a second slot running from said third opening in said housing to allow drop-in insertion of said outlet tube without requiring detachment of said outlet tube from distal attachments.

3. The device of claim 2, and further comprising:
An open-close lid on said housing at said opening, wherein said lid is openable to allow drop-in insertion of said squeeze bag within said housing and closable to confine said squeeze bag within said housing.

4. The device of claim 3, and further comprising:
A first yoke near said first opening to confine a first end of said squeeze bag; and,
A second yoke near said second opening to confine a second, opposite end of said squeeze bag.

5. The device of claim 4, and further comprising:
an adjustable volume controller providing operator adjustment of the magnitude of squeezing by said mechanical compression squeezer to adjust the volume of air/gas per cycle through said AMBU-bag; and,
an adjustable cycle controller providing operator adjustment of cycle frequency of said mechanical compression squeezer.

6. The device of claim 5 and further including a built-in electronic blood-oxygen level sensor.

7. The device of claim 6 and further including a built-in CO₂ meter.

8. The device of claim 7 wherein said housing includes at least two recessable hooks adapted to hang said housing off a hospital bed rail.

9. The device of claim 8 wherein said housing is portable such that it will fit in a vehicle, such as a helicopter or ambulance, and includes a battery for providing power to said powered actuator.

10. The device of claim 9 wherein said mechanical compression squeezer includes a movable rigid member opposite of a fixed portion of said housing with said squeeze bag therebetween.

11. The device of claim 9 wherein said mechanical compression squeezer includes a flexible tension member that pulls hoop stress on an outside of said squeeze bag.

12. The device of claim 1, and further comprising:
An open-close lid on said housing, wherein said lid is openable to allow drop-in insertion of said squeeze bag within said housing and closable to confine said squeeze bag within said housing.

13. The device of claim 1, and further comprising:
A first yoke near said first opening to confine a first end of said squeeze bag; and,
A second yoke near said second opening to confine a second, opposite end of said squeeze bag.

14. The device of claim 1, and further comprising:
an adjustable volume controller providing operator adjustment of the magnitude of squeezing by said mechanical compression squeezer to adjust the volume of air/gas per cycle through said AMBU-bag; and,
an adjustable cycle controller providing operator adjustment of cycle frequency of said mechanical compression squeezer.

15. The device of claim 1 and further including a built-in electronic blood-oxygen level sensor.

16. The device of claim 1 and further including a built-in CO₂ meter.

17. The device of claim 1 wherein said housing includes at least two recessable hooks adapted to hang said housing off a hospital bed rail.

18. The device of claim 1 wherein said housing is portable such that it will fit in a vehicle, such as a helicopter or ambulance, and includes a battery for providing power to said powered actuator.

19. The device of claim 1 wherein said mechanical compression squeezer includes a movable rigid member opposite of a fixed portion of said housing with said squeeze bag therebetween.

20. The device of claim 1 wherein said mechanical compression squeezer includes a flexible tension member that pulls hoop stress on an outside of said squeeze bag.

21. A device for use with an AMBU-bag having a flexible squeeze bag, comprising:
a housing for receiving said squeeze bag;
a mechanical compression squeezer in said housing for cyclically squeezing said squeeze bag from its outside and releasing said squeeze bag for expansion; and
a powered actuator for powering said mechanical compression squeezer for said cyclical squeezing; and,
wherein said housing includes at least two recessable hooks adapted to hang said housing off a hospital bed rail.

22. A device for use with an AMBU-bag having a flexible squeeze bag, comprising:
a housing for receiving said squeeze bag;
a mechanical compression squeezer in said housing for cyclically squeezing said squeeze bag from its outside and releasing said squeeze bag for expansion; and, a powered actuator for powering said mechanical compression squeezer for said cyclical squeezing
wherein said housing is portable such that it will fit in a vehicle, such as a helicopter or ambulance, and includes a battery for providing power to said powered actuator.
